(19) United States
(12) Patent Application Publication
(10) Pub. No.: US 2015/0232426 A1
(43) Pub. Date: Aug. 20, 2015

(54) PROCESS FOR THE PREPARATION OF OXCARBAZEPINE AND ITS USE AS INTERMEDIATE IN THE PREPARATION OF ESLICARBAZEPINE ACETATE

(71) Applicant: RANBAXY LABORATORIES LIMITED, New Delhi, Delhi (IN)

(72) Inventors: Ketan Hirpara, Junagadh (IN); Kdv Jesunadh, East Godavari (IN); Mukesh Kumar Sharma, Gurgaon (IN); Chandra Has Khanduri, Gurgaon (IN)

(21) Appl. No.: 14/428,418

(22) PCT Filed: Sep. 26, 2013
(86) PCT No.: PCT/IB2013/058892

§ 371 Date: Mar. 16, 2015

(30) Foreign Application Priority Data
Sep. 26, 2012 (IN) 3003/DEL/2012

(51) Int. Cl. C07D 223/28

(52) U.S. Cl. CPC C07D 223/28

(57) ABSTRACT

The present invention provides a process for the preparation of oxcarbazepine of Formula (1), which is an Active Pharmaceutical Ingredient (API) and a useful intermediate in the preparation of eslicarbazepine acetate of Formula (A). The present invention further provides a process for the preparation of eslicarbazepine acetate.

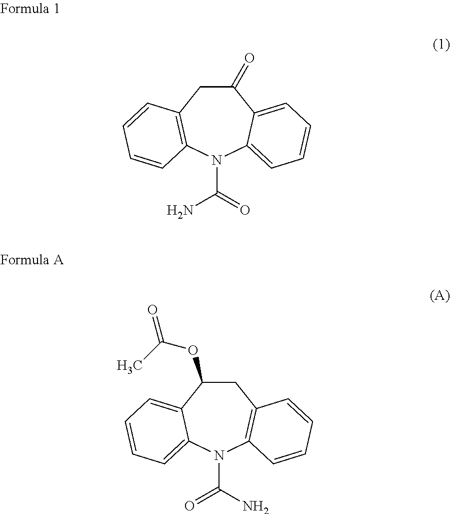 [SMILES: NC(=O)N1c2ccccc2CC(=O)c2ccccc21 ; CC(=O)O[C@H]1Cc2ccccc2N(C(N)=O)c2ccccc21]

### FIELD OF THE INVENTION

The present invention provides a process for the preparation of oxcarbazepine, which is an Active Pharmaceutical Ingredient (API), and a useful intermediate in the preparation of eslicarbazepine acetate. The present invention further provides a process for the preparation of eslicarbazepine acetate.

### BACKGROUND OF THE INVENTION

10-oxo-10,11-dihydro-5H-dibenzo[b,f]azepine-5-carboxamide of Formula 1, commonly known as oxcarbazepine, is an antiepileptic drug marketed under the trade name Trileptal®. Oxcarbazepine is also an intermediate in the preparation of eslicarbazepine acetate.

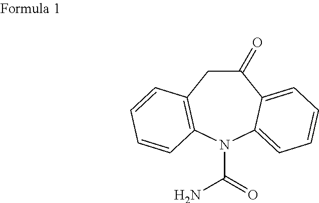 [SMILES: NC(=O)N1c2ccccc2CC(=O)c2ccccc21]

U.S. Pat. No. 3,642,775 provides a process for the preparation of oxcarbazepine which involves refluxing 10-methoxy-5H-dibenz[b,f]azepine-5-carboxamide with 2N hydrochloric acid for 2 hours. Oxcarbazepine was isolated from the reaction mixture by cooling, filtering, and finally recrystallizing from ethanol with a yield of 80%.

PCT Publication No. WO 96/21649 provides a process for the preparation of oxcarbazepine which involves refluxing 10-methoxy-5H-dibenz[b,f]azepine-5-carboxamide with 10% sulfuric acid for 2 hours to 3 hours. Oxcarbazepine was isolated from the reaction mixture by cooling, filtering, washing with water, and finally recrystallizing from dimethylacetamide.

PCT Publication No. WO 01/56992 provides a process for the preparation of oxcarbazepine which involves adding water and 100% sulfuric acid to a mixture of 10-methoxy-5H-dibenzo[b,f]azepine-5-carboxamide in acetic acid until the pH is less than 1, and then stirring the reaction mixture for 17 hours. Oxcarbazepine was isolated from the reaction mixture by adding water and filtering the precipitated oxcarbazepine, with a yield of 78%.

PCT Publication No. WO 02/096881 provides a process for the preparation of oxcarbazepine which involves oxidation of 10,11-dihydro-10-hydroxy-5H-dibenzo[b,f]azepine-5-carboxamide with peroxyacetic acid in the presence of potassium dichromate adsorbed on silica gel at room temperature.

U.S. Pat. No. 7,459,553 (herein after referred to as U.S. '553) provides a process for the preparation of oxcarbazepine which involves adding 2N hydrochloric acid to 10-methoxycarbamazepine in dichloromethane and maintaining the reaction mixture at about 40° C. to 50° C. for about 4 hours to 6 hours. Oxcarbazepine was isolated from the reaction mixture by cooling the reaction mixture to 0° C. to 5° C. and filtering the separated solid. Another process for the preparation of oxcarbazepine involves adding 2N hydrochloric acid to 10-methoxycarbamazepine and maintaining the reaction mixture at about 80° C. to 85° C. for about 4 hours to 5 hours. Oxcarbazepine was isolated from the reaction mixture by cooling the reaction mixture to 50° C., adding toluene and maintaining the reaction mixture at 50° C. for 30 minutes, then further cooling the reaction mixture to about 25° C. to 30° C. and filtering the solid. The oxcarbazepine obtained by the second process provided in U.S. '553 has a purity of 97% to 98% and requires further purification.

U.S. Pat. No. 6,670,472 (herein after referred to as U.S. '472) provides a process for the preparation of oxcarbazepine which involves treating 10-methoxyiminostilbene with either benzoic acid and sodium cyanate, p-chlorobenzoic acid and sodium cyanate, 2,4-dichloro benzoic acid and sodium cyanate or benzoic acid and potassium cyanate, the product of which was then hydrolyzed with either 2N hydrochloric acid, 2N sulphuric acid, or 2N monochloroacetic acid to obtain oxcarbazepine. The oxcarbazepine thus obtained by the processes provided in U.S. '472 involves further purification in a mixture of dichloromethane:methanol, dichloromethane:toluene or toluene:methanol.

PCT Publication No. WO 2005/066133 provides a process for the preparation of oxcarbazepine which involves dissolving 10-methoxy-5H-dibenzo[b,f]azepine-5-carboxamide in ethylene dichloride, adding o-toluene sulfonic acid and maintaining the reaction mixture at about 75° C. to 80° C. for about 3 hours. Oxcarbazepine was isolated from the reaction mixture by cooling the reaction mixture to 20° C., filtering, and further purifying using acetone-water.

PCT Publication No. WO 2005/092862 provides a process for the preparation of oxcarbazepine which involves adding water to a stirred suspension of 10-methoxy-5H-dibenzo[b,f]azepine-5-carboxamide and 37% hydrochloric acid at pH 1 and stirring at 95° C. for 4 hours. Oxcarbazepine was isolated from the reaction mixture by cooling to 25° C., adding 30% sodium hydroxide until pH was 7.0 to 7.5, filtering, and washing with water. The oxcarbazepine thus obtained was again purified to attain a purity of 99%.

EP Publication No. EP 1600443 provides a process for the preparation of oxcarbazepine which involves hydrolysis of 10-methoxy-N-aminocarbonyl-iminostilbene by refluxing with 10% sulfuric acid for one hour. Oxcarbazepine was isolated from the reaction mixture by cooling to room temperature, filtering, washing with water, and further recrystallizing from dimethylformamide.

PCT Publication No. WO 2007/141798 provides a process for the preparation of oxcarbazepine which involves hydrolysis of 10-methoxy-5H-dibenzo[b,f]azepine-5-carboxamide in toluene and water with concentrated hydrochloric acid at a temperature of 75° C. to 80° C. Oxcarbazepine was isolated from the reaction mixture by cooling the reaction mixture, filtering, washing with toluene, and washing with 5% sodium bicarbonate and water. The oxcarbazepine thus obtained was purified twice using methanol and methanol/dichloromethane, respectively.

PCT Publication No. WO 2009/139001 provides a process for the preparation of oxcarbazepine which involves heating 10-methoxycarbamazepine in water and oxalic acid at 90° C. for about 17 hours. Oxcarbazepine was isolated from the reaction mixture by cooling the reaction mixture to room temperature, filtering, washing with water, and further purifying using isopropyl alcohol and water.

Several other processes are known in the literature for making oxcarbazepine, for example, U.S. Pat. No. 4,452,738; and PCT Publication Nos. WO 2010/000196, WO 2008/012837, WO 2006/075925, WO 2005/122671, WO 2005/118550, WO 2005/096709, WO 03/106414, and WO 00/55138; and Organic Process Research & Development, 9(3), 272-277 (2005).

Eslicarbazepine acetate of Formula A, chemically known as (10S)-5-carbamoyl-10,11-dihydro-5H-dibenzo[b,f]azepin-10-yl acetate is indicated as adjunctive therapy in adults with partial-onset seizures with or without secondary generalisation.

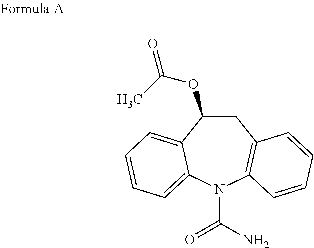 [SMILES: CC(=O)O[C@H]1Cc2ccccc2N(C(N)=O)c2ccccc21]

Several processes are known in the literature for making and purifying eslicarbazepine acetate, for example, PCT Publication Nos. WO 2006/005951, WO 2007/117166, and WO 2010/113179, Journal of Medicinal Chemistry, 42(14), 2582-2587 (1999).

U.S. Pat. No. 5,753,646 provides a process for the preparation of eslicarbazepine acetate which involves the drop-wise addition of a solution of acetyl chloride in dichloromethane to a suspension of (−)-10-hydroxy-10,11-dihydro-5H-dibenzo[b,f]azepine-5-carboxamide in dichloromethane and pyridine at a temperature of less than 10° C. under stirring. The residue obtained after work up was crystallized from a mixture of dichloromethane and ethyl acetate to give the eslicarbazepine acetate as white crystals.

PCT Publication No. WO 02/092572 provides a process for the preparation of eslicarbazepine which involves optical resolution of racemic (±)-10,11-dihydro-10-hydroxy-5H-dibenzo[b,f]azepine-5-carboxamide using diacetyl tartaric anhydride.

PCT Publication No. WO 2004/031155 (herein after WO '155) provides a process for the preparation of eslicarbazepine which involves enantioselective transfer hydrogenation of oxcarbazepine using triethyl amine and formic acid in the presence of RuCl[(1S,2S)-p-TsNCH(C₆H₅)CH(C₆H₅)NH₂](η⁶-p-cymene) in dichloromethane at reflux temperature. The present inventors observed that the process provided in WO '155 leads to degradation and only 60% to 70% of the reaction is completed in 17 hours. Further, the process requires flash chromatography to isolate eslicarbazepine from the reaction mixture. Thus, the process is not commercially viable.

PCT Publication No. WO 2006/056339 provides a process for the preparation of eslicarbazepine which involves hydrolysis of (S)-(+)-5-cyano-10,11-dihydro-10-hydroxy-5H-dibenzo[b,f]azepine using peroxy compounds such as sodium perborate or hydrogen peroxide in alkaline medium.

PCT Publication No. WO 2007/012793 provides a process for the preparation of eslicarbazepine which involves asymmetric reduction of oxcarbazepine using triethylamine and formic acid at a pH range of 6.5 to 8 in the presence of a catalyst generated in situ by the reaction of RuCl₂(p-cymene)]₂ and (S,S)-TsDAEN.

PCT Publication No. WO 2011/091131 provides a process for the preparation of eslicarbazepine which involves optical resolution of racemic (±)-10,11-dihydro-10-hydroxy-5H-dibenzo[b,f]azepine-5-carboxamide using naproxen. It also provides another process for the preparation of eslicarbazepine which involves asymmetric reduction of oxcarbazepine using a borane dimethyl sulfide complex in the presence of R-MeCBS.

PCT Publication No. WO 2011/131315 provides processes for the preparation of eslicarbazepine which involve asymmetric transfer hydrogenation of oxcarbazepine in the presence of catalysts such as, RuCl[(S,S)-Ts-DPEN](p-cymene), RuCl[(S,S)-Ms-DPEN](p-cymene), RuCl[(S,S)-teth-TsDPEN], RuCl[(S,S)-Fs-DPEN](p-cymene) and an ion exchange resin such as IRA-67 tertiary ion exchange resin. An alternative process for the preparation of eslicarbazepine involves asymmetric transfer hydrogenation of oxcarbazepine in the presence of catalyst RuCl[(S,S)-Ts-DPEN](p-cymene) and a quaternary amine such as tetramethylammonium hydroxide.

Journal of Molecular Catalysis B: Enzymatic, 72, 294-297 (2011) provides a process for the preparation of eslicarbazepine which involves asymmetric reduction of oxcarbazepine with an enzyme, Saccharomyces cerevisiae CGMCC No. 2266. According to this publication, the optimum reaction time was 36 hours, optimum reaction temperature was 30° C., optimum initial reaction pH was 7, and a continuous reduction method was preferred to obtain eslicarbazepine.

Journal of American Chemical Society, 118(10), 2521-2522 (1996) provides the use of ruthenium complexes such as [(S,S)-TsDpen-Ru(p-cymene)Cl] as catalysts and triethylamine/formic acid as a hydrogen donor for the enantioselective reduction of simple ketones.

The present inventors identified that oxcarbazepine prepared by the hydrolysis of 10-methoxy-5H-dibenzo[b,f]azepine-5-carboxamide using hydrochloric acid, sulfuric acid, or acetic acid results in a low yield and chromatographic purity. Therefore, further purification of oxcarbazepine would be necessary to improve the quality of the product. Further reagents such as hydrochloric acid, sulfuric acid, and acetic acid are corrosive in nature and are therefore undesirable.

Extensive experimentation has been carried out by controlling parameters such as the combination of solvents in various ratios, reagents/catalysts which are highly efficient, commercially available, and economically feasible for developing an improved process for the preparation of oxcarbazepine and eslicarbazepine.

Thus, the present invention provides an efficient, industrially preferable, and economic process for preparing oxcarbazepine and eslicarbazepine in good yield with excellent chemical and enantiomeric purity. The present invention further provides eslicarbazepine acetate in good yield, with excellent chemical and enantiomeric purity.

The present inventors have developed an improved process for the preparation of oxcarbazepine and eslicarbazepine which avoids the excess usage of environmentally hazardous reagents and organic solvents, thereby promoting green chemistry and ensuring cleaner surroundings by putting a lesser load on the environment.

### SUMMARY OF THE INVENTION

One aspect of the present invention provides a process for the preparation of eslicarbazepine acetate of Formula A

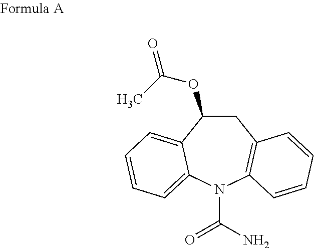 [SMILES: CC(=O)O[C@H]1Cc2ccccc2N(C(N)=O)c2ccccc21]

which comprises:
a) hydrolysis of 10-methoxy-5H-dibenzo[b,f]azepine-5-carboxamide of Formula 3

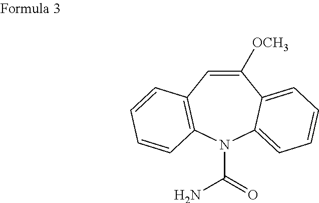 [SMILES: COC1=Cc2ccccc2N(C(N)=O)c2ccccc21]

with an organic acid selected from the group consisting of citric acid, tartaric acid, or mixtures thereof to obtain oxcarbazepine of Formula 1;

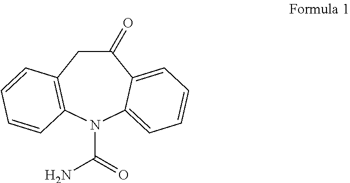 [SMILES: NC(=O)N1c2ccccc2CC(=O)c2ccccc21]

b) asymmetric transfer hydrogenation of the oxcarbazepine of Formula 1 in the presence of a catalyst and a hydride source in a mixture of dichloromethane/N,N-dimethylformamide, dichloromethane/water, or dichloromethane/water/methanol to obtain eslicarbazepine of Formula 2; and

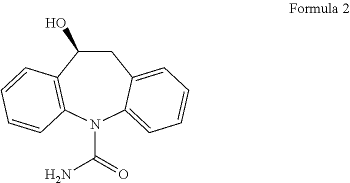 [SMILES: NC(=O)N1c2ccccc2C[C@H](O)c2ccccc21]

c) acylation of the eslicarbazepine of Formula 2 to obtain eslicarbazepine acetate of Formula A.

Another aspect of the present invention provides a process for the preparation of oxcarbazepine of Formula 1

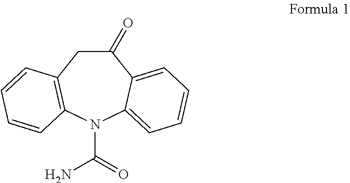 [SMILES: NC(=O)N1c2ccccc2CC(=O)c2ccccc21]

which comprises hydrolysis of 10-methoxy-5H-dibenzo[b,f]azepine-5-carboxamide of Formula 3

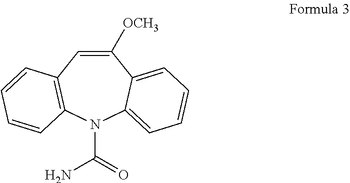 [SMILES: COC1=Cc2ccccc2N(C(N)=O)c2ccccc21]

with an organic acid selected from the group consisting of citric acid, tartaric acid, or mixtures thereof.

### DETAILED DESCRIPTION OF THE INVENTION

One aspect of the present invention provides a process for the preparation of eslicarbazepine acetate of Formula A

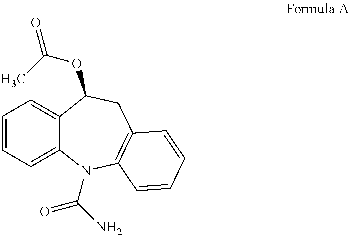 [SMILES: CC(=O)O[C@H]1Cc2ccccc2N(C(N)=O)c2ccccc21]

which comprises:
a) hydrolysis of 10-methoxy-5H-dibenzo[b,f]azepine-5-carboxamide of Formula 3

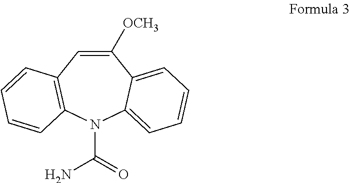 [SMILES: COC1=Cc2ccccc2N(C(N)=O)c2ccccc21]

with an organic acid selected from the group consisting of citric acid, tartaric acid, or mixtures thereof to obtain oxcarbazepine of Formula 1;

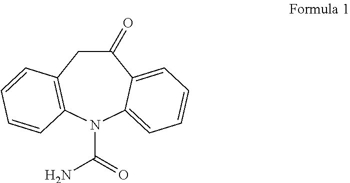 [SMILES: NC(=O)N1c2ccccc2CC(=O)c2ccccc21]

b) asymmetric transfer hydrogenation of the oxcarbazepine of Formula 1 in the presence of a catalyst and a hydride source in a mixture of dichloromethane/N,N-dimethylformamide, dichloromethane/water, or dichloromethane/water/methanol to obtain eslicarbazepine of Formula 2; and

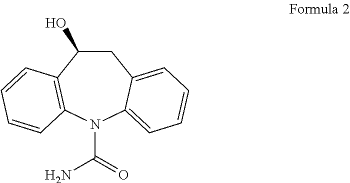 [SMILES: NC(=O)N1c2ccccc2C[C@H](O)c2ccccc21]

c) acylation of eslicarbazepine of Formula 2 to obtain eslicarbazepine acetate of Formula A.

In one embodiment of this aspect, step a) is performed in one or more solvent.

The “solvent” is selected from the group consisting of water, esters, aromatic hydrocarbons, halogenated hydrocarbons, ketones, ethers, polar aprotic solvents, or mixtures thereof.

Examples of esters include ethyl acetate, n-propyl acetate, isopropyl acetate, and n-butyl acetate. Examples of aromatic hydrocarbons include toluene and xylene. Examples of halogenated hydrocarbons include dichloromethane, chloroform, and 1,2-dichloroethane. Examples of ketones include acetone and methyl ethyl ketone. Examples of ethers include diethyl ether and tetrahydrofuran. Examples of polar aprotic solvents include N,N-dimethylformamide, N,N-dimethylacetamide, dimethylsulphoxide, acetonitrile, and N-methylpyrrolidone.

The hydrolysis of 10-methoxy-5H-dibenzo[b,f]azepine-5-carboxamide of Formula 3 in step a) is performed at a temperature of 30° C. to reflux for a time period sufficient to complete the reaction. After completion of the reaction, oxcarbazepine may be isolated by a common isolation technique such as cooling, extraction, washing, crystallization, precipitation, filtration, filtration under vacuum, decantation and centrifugation, or combinations thereof.

The isolated oxcarbazepine may be optionally purified by crystallization or chromatographic methods, or combinations thereof, before proceeding to step b).

In one embodiment of this aspect, step b) may be performed optionally in the presence of a phase transfer catalyst.

A suitable phase transfer catalyst may be tetra-n-butylammonium bromide.

The catalyst is selected from the group consisting of [(S,S)-TsDpen-Ru(p-cymene)Cl], [(S,S)-teth-TsDpen-RuCl], RuCl[(S,S)-FsDPEN](p-cymene), RuCl[(S,S)-TsDPEN](mesitylene). The molar ratio of the catalyst to oxcarbazepine may be from about 0.0005 to about 0.1.

The hydride source is selected from the group consisting of sodium acetate/water, formic acid/triethyl amine, potassium-t-butoxide/isopropanol, potassium hydroxide/isopropanol, ammonium formate, and ammonium acetate. The molar ratio of the hydride source to oxcarbazepine may be from about 0.1 to about 8.

After the completion of the reaction, eslicarbazepine may be isolated by a common isolation technique such as cooling, extraction, washing, crystallization, precipitation, filtration, filtration under vacuum, decantation and centrifugation, or combinations thereof.

The isolated eslicarbazepine may be optionally purified by crystallization or chromatographic methods, or combinations thereof, before proceeding to step c).

In another embodiment of this aspect, acylation of eslicarbazepine of Formula 2 in step c) involves treating eslicarbazepine with an acylating agent in the presence of a catalyst in one or more solvents at a temperature of 25° C. to reflux for a time period sufficient to complete the reaction.

The term “treating” includes adding, dissolving, slurrying, stirring, or combinations thereof.

The acylating agent is selected from the group consisting of acetic anhydride or acetyl chloride. A suitable catalyst may be 4-Dimethylaminopyridine.

After completion of the reaction, eslicarbazepine acetate of Formula A may be optionally isolated by a common isolation technique such as cooling, extraction, washing, crystallization, precipitation, filtration, filtration under vacuum, decantation and centrifugation, or combinations thereof.

The isolated eslicarbazepine acetate may be optionally purified by crystallization or chromatographic methods, or combinations thereof.

Another aspect of the present invention provides a process for the preparation of oxcarbazepine of Formula 1,

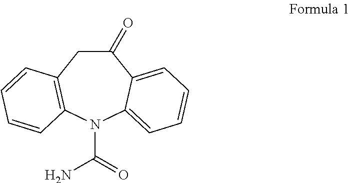 [SMILES: NC(=O)N1c2ccccc2CC(=O)c2ccccc21]

which comprises hydrolysis of 10-methoxy-5H-dibenzo[b,f]azepine-5-carboxamide of Formula 3

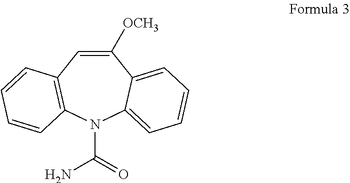 [SMILES: COC1=Cc2ccccc2N(C(N)=O)c2ccccc21]

with an organic acid selected from the group consisting of citric acid, tartaric acid, or mixtures thereof.

In one embodiment of this aspect, the hydrolysis of 10-methoxy-5H-dibenzo[b,f]azepine-5-carboxamide of Formula 3 is performed in one or more solvents.

The “solvent”, as used herein, has the same meaning as defined above.

The hydrolysis of 10-methoxy-5H-dibenzo[b,f]azepine-5-carboxamide of Formula 3 is performed at a temperature of 30° C. to reflux temperature for a time period sufficient to complete the reaction.

After the completion of the reaction, oxcarbazepine can be isolated by a common isolation technique such as cooling, extraction, washing, crystallization, precipitation, filtration, filtration under vacuum, decantation and centrifugation, or combinations thereof.

The isolated oxcarbazepine may be optionally purified by crystallization or chromatographic methods, or combinations thereof.

The oxcarbazepine obtained according to the present invention may be used as an Active Pharmaceutical Ingredient (API) and may be formulated into finished pharmaceutical products. Alternatively, it can be converted to eslicarbazepine or eslicarbazepine acetate by the methods exemplified herein or methods known in the art.

While the present invention has been described in terms of its specific embodiments, certain modifications and equivalents will be apparent to those skilled in the art and are intended to be included within the scope of the present invention.

### EXAMPLES

### Example 1

### Preparation of oxcarbazepine

10-methoxy-5H-dibenzo[b,f]azepine-5-carboxamide (2 g, 0.0075 mol) was added to 10% aqueous tartaric acid (20 mL) at 25° C. to 30° C. under stirring. The reaction mixture was heated to 95° C. to 100° C. and stirred for 2 hours. The reaction mixture was then cooled to 25° C. to 30° C. and stirred at 25° C. to 30° C. for 3 hours. The solid was filtered, washed with deionized water (20 mL), and dried at 50° C. to 55° C. until constant weight was achieved to obtain the title compound.

Yield: 1.7 g (90.42%)

Chromatographic Purity: 99.39%

### Example 2

### Preparation of Oxcarbazepine

10-methoxy-5H-dibenzo[b,f]azepine-5-carboxamide (1 g, 0.00375 mol) was added to 5% aqueous citric acid (10 mL) at 25° C. to 30° C. under stirring. The reaction mixture was heated to 95° C. to 100° C. and stirred for 2 hours. The reaction mixture was then cooled to 25° C. to 30° C. and deionized water (15 mL) was added and stirred at 25° C. to 30° C. for 3 hours. The solid was filtered, washed with deionized water (10 mL), and dried at 50° C. to 55° C. until constant weight was achieved to obtain the title compound.

Yield: 0.88 g (93.6%)

Chromatographic Purity: 99.24%

### Example 3

### Preparation of Oxcarbazepine

10-methoxy-5H-dibenzo[b,f]azepine-5-carboxamide (1 g, 0.00375 mol) was added to 5% aqueous acetic acid (10 mL) at 25° C. to 30° C. under stirring. The reaction mixture was heated to 95° C. to 100° C. and stirred for 3 hours. The reaction mixture was then cooled to 25° C. to 30° C., and the solid was filtered, washed with deionized water (10 mL), and dried at 50° C. to 55° C. until constant weight was achieved to obtain the title compound.

Yield: 0.81 g (85.2%)

Chromatographic Purity: 98.85%

### Example 4

### Preparation of Oxcarbazepine

10-methoxy-5H-dibenzo[b,f]azepine-5-carboxamide (5 g, 0.0187 mol) was charged to 5% aqueous citric acid (50 mL; 2.5 g citric acid in 50 mL water). Toluene (25 mL) was charged to the reaction mixture and heated to reflux (86° C.). The reaction mixture was stirred for 12 hours at 86° C. The reaction mixture was cooled to 25° C. to 30° C. and stirred for 2 hours. The solid was filtered and dried for 2 hours at 50° C. to 55° C. to obtain the title compound.

Yield: 4.4 g (93%)

Chromatographic Purity: 99.72% (0.12% unreacted 10-methoxy carbamazepine)

### Example 5

### Preparation of Oxcarbazepine

2N aqueous hydrochloric acid (50 mL) was added to 10-methoxy-5H-dibenzo[b,f]azepine-5-carboxamide (5.0 g, 0.0187 mol) at 25° C. to 30° C. and was stirred for 10 minutes. The reaction mixture was heated to 95° C. to 100° C. for 2 hours. After completion of the reaction, the reaction mixture was cooled to 10° C. to 15° C. and stirred for 15 minutes to 20 minutes. The solid was filtered and washed with water (15 mL). The solid was suck dried and further dried at 50° C. to 55° C. under vacuum for 15 hours to obtain crude oxcarbazepine.

Yield: 4.3 g (90.15%)

Chromatographic Purity: 96.53%

The crude oxcarbazepine was suspended in denatured spirits (10 mL) and the resulting slurry was heated to reflux for 30 minutes. The suspension was cooled to 0° C. to 5° C. and stirred for 1 hour. The solid was isolated by filtration and dried under vacuum to obtain the title compound.

Yield: 3.7 g (77.56%)

Chromatographic Purity: 97.89%

### Example 6

### Preparation of Eslicarbazepine

[(S,S)-TsDpen-Ru(p-cymene)Cl] (52 mg, 0.000081 mol) was added to a stirred suspension of oxcarbazepine (5.0 g, 0.01983 mol) and dichloromethane (200 mL) at 25° C. to 30° C. under nitrogen followed by the drop-wise addition of a premixed solution of formic acid and triethyl amine (4.8 mL:8 mL) in dichloromethane (50 mL). After stirring at 25° C. to 30° C. for 15 minutes, the reaction mixture was heated to reflux (39° C. to 42° C.) for 4 days. The pH of the reaction mixture was maintained at 5.4 to 6.5 by the addition of formic acid (0.8 mL). After completion of the reaction, the reaction mixture was concentrated under reduced pressure to 50 mL and hexane (200 mL) was charged dropwise and stirred for 3 hours at 25° C. to 30° C. The reaction mixture was filtered and washed with hexane (10 mL). The solid obtained was suck dried and further dried at 50° C. to 55° C. for 5 hours to obtain the title compound.

Yield: 4.4 g (88%)

Chromatographic Purity: 99.58%

Chiral purity: 99.4%

### Example 7

### Preparation of Eslicarbazepine Acetate

4-Dimethylaminopyridine (0.06 g, 0.00047 mol) was added to a stirred suspension of the eslicarbazepine obtained in Example 6 (2.0 g, 0.0079 mol) in acetone (12 mL) at 25° C. to 30° C. The reaction mixture was stirred at 25° C. to 30° C. and acetic anhydride (1.1 g, 0.0102 mol) was added to it. The reaction mixture was stirred at 25° C. to 30° C. for 2 hours. After completion of the reaction, deionized water (36 mL) was charged drop-wise and stirred for 3 hours at 18° C. to 20° C. The reaction mixture was filtered, then washed with deionized water (4 mL). The solid thus obtained was suck dried and further dried at 50° C. to 55° C. for 12 hours to obtain the title compound.

Yield: 1.81 g (78.7%)

Chromatographic Purity: 99.41%,

Chiral purity: 99.66%

### Example 8

### Preparation of Eslicarbazepine

[(S,S)-TsDpen-Ru(p-cymene)Cl] (50 mg, 0.000078 mol) was added to a stirred suspension of oxcarbazepine (5.0 g, 0.01983 mol), dichloromethane (75 mL), and N,N-dimethylformamide (10 mL) at 25° C. to 30° C. under nitrogen followed by drop-wise addition of a premixed solution of formic acid and triethyl amine (5.6 mL:8 mL) in dichloromethane (50 mL). After stirring at 25° C. to 30° C. for 15 minutes, the reaction mixture was heated to reflux (39° C. to 42° C.) for 18 hours. The pH was maintained at 5.4 to 6.5 by the addition of formic acid (0.5 mL). After completion of the reaction, the reaction mixture was washed with aqueous sodium bicarbonate (50 mL). The organic layer was evaporated completely under reduced pressure at 40° C. to 42° C. to obtain the title compound.

Yield: 4.3 g (86%)

Chromatographic Purity: 98.21%

Chiral purity: 100%

### Example 9

### Preparation of Eslicarbazepine

Oxcarbazepine (1.0 g, 0.00396 mol), aqueous sodium formate (27 g, 0.396 mol in 80 mL deionized water), and tetra-n-butylammonium bromide (3 g, 0.0093 mol) were added to a stirred suspension of [(S,S)-TsDpen-Ru(p-cymene)Cl] (10 mg, 0.000014 mol), dichloromethane (8 mL), and triethyl amine (10 mg, 0.000099 mol) at 25° C. to 30° C. under nitrogen. After stirring at 25° C. to 30° C. for 15 minutes, the reaction mixture was heated to 38° C. to 40° C. overnight. After completion of the reaction, the reaction mixture was washed with aqueous sodium bicarbonate (50 mL) and brine (50 mL). The organic layer was evaporated completely under reduced pressure at 40° C. to 42° C. The residue was purified by column chromatography using dichloromethane as eluent to obtain the title compound.

Yield: 0.58 g

Chromatographic Purity: 95.03%

Chiral purity: 99.51%

### Example 10

### Preparation of Eslicarbazepine Acetate

Acetone (6 mL) and 4-dimethylaminopyridine (0.028 g, 0.0000235 mol) were added to a stirred suspension of the eslicarbazepine obtained by Example 9 (1.1 g, 0.00393 mol) at 25° C. to 30° C. The reaction mixture was stirred at 25° C. to 30° C. and acetic anhydride (0.6 g, 0.0051 mol) was added. The reaction mixture was stirred at 25° C. to 30° C. for 2 hours. After completion of the reaction, deionized water (22 mL) was charged drop-wise and stirred for 3 hours at 10° C. to 15° C. The reaction mixture was filtered and washed with deionized water (15 mL). The solid thus obtained was suck dried and further dried at 50° C. to 55° C. for 3 hours to obtain the title compound.

Yield: 0.45 g (35.4%)

Chromatographic Purity: 99.38%

Chiral purity: 99.7%

### Example 11

### Preparation of Eslicarbazepine

Formic acid and triethyl amine (3.3 g:2.9 g) were added to a stirred suspension of oxcarbazepine (3.0 g, 0.0119 mol) and dichloromethane (45 mL) at 25° C. to 30° C. under nitrogen. [(S,S)-TsDpen-Ru(p-cymene)Cl] (30 mg, 0.000047 mol) in N,N-dimethylformamide (5.4 mL) was added to the reaction mixture. After stirring at 25° C. to 30° C. for 15 minutes, the reaction mixture was heated to reflux (39° C. to 42° C.) for 20 hours. After completion of the reaction, the reaction mixture was evaporated under reduced pressure (500 mm/Hg to 700 mm/Hg) at 40° C. to 42° C. Deionized water (60 mL) was charged drop-wise and stirred for 2 hours at 0° C. to 5° C. The reaction mixture was filtered and washed with deionised water (20 mL). The solid obtained was suck dried and further dried at 50° C. to 55° C. for 12 hours to obtain the title compound.

Yield: 2.53 g (84.3%)

Chromatographic Purity: 98.21%

Chiral purity: 100%

### Example 12

### Preparation of Eslicarbazepine Acetate

Acetone (6 mL) and 4-dimethylaminopyridine (0.048 g, 0.00004 mol) were added to a stirred suspension of the eslicarbazepine obtained by Example 11 (1.5 g, 0.0059 mol) at 25° C. to 30° C. The reaction mixture was stirred at 25° C. to 30° C. and acetic anhydride (0.8 g, 0.0071 mol) was added. The reaction mixture was stirred at 25° C. to 30° C. for 2 hours. After completion of the reaction, deionized water (27 mL) was charged drop-wise and stirred for 3 hours at 0° C. to 5° C. The reaction mixture was filtered and washed with deionized water (15 mL). The solid obtained was suck dried and further dried at 50° C. to 55° C. for 3 hours to obtain the title compound.

Yield: 1.45 g (83.3%)

Chiral purity: 99.9%

### Example 13

### Preparation of Eslicarbazepine

Formic acid and triethyl amine (2.2 g: 1.9 g) were added to a stirred suspension of oxcarbazepine (2.0 g, 0.0079 mol) and dichloromethane (30 mL) at 25° C. to 30° C. under nitrogen. [(S,S)-TsDpen-Ru(p-cymene)Cl] (20 mg, 0.000031 mol) in methanol (10 mL), tetra-n-butylammonium bromide (200 mg), and water (2 mL) were added to the reaction mixture. After stirring at 25° C. to 30° C. for 15 minutes, the reaction mixture was heated to reflux (39° C. to 42° C.) for 24 hours. After completion of the reaction, the solvent was completely evaporated under reduced pressure (500 mm/Hg to 700 mm/Hg) at 45° C. to 47° C. Deionized water (30 mL) was charged drop-wise and stirred for 1 hour at 0° C. to 5° C. The reaction mixture was filtered and washed with deionized water (20 mL). The solid thus obtained was suck dried and further dried at 50° C. to 55° C. for 12 hours to obtain the title compound.

Yield: 1.65 g (81.88%)

Chromatographic Purity: 99.76%

Chiral purity: 100%

### Example 14

### Preparation of Eslicarbazepine

Formic acid and triethyl amine (2.2 g:1.9 g) were added to a stirred suspension of oxcarbazepine (2.0 g, 0.0079 mol) and dichloromethane (30 mL) at 25° C. to 30° C. under nitrogen. [(S,S)-TsDpen-Ru(p-cymene)Cl] (20 mg, 0.000031 mol) in ethyl acetate (10 mL), tetra-n-butylammonium bromide (200 mg), and water (2 mL) were added to the reaction mixture. After stirring at 25° C. to 30° C. for 15 minutes, the reaction mixture was heated to reflux for 30 hours. After completion of the reaction, the solvent was completely evaporated under reduced pressure (500 mm/Hg to 700 mm/Hg) at 40° C. to 42° C. Deionized water (30 mL) was charged drop-wise and stirred for 2 hours at 0° C. to 5° C. The reaction mixture was filtered and washed with deionized water (20 mL). The solid thus obtained was suck dried and further dried at 50° C. to 55° C. for 12 hours to obtain the title compound.

Yield: 1.73 g (85.8%)

Chromatographic Purity: 99.76%

Chiral purity: 100%

### Example 15

### Preparation of Eslicarbazepine

A solution of triethyl amine (33.1 g, 0.327 mol) in dichloromethane (50 mL) was slowly added to a stirred solution of formic acid (15.05 g, 0.327 mol) in dichloromethane (1600 mL) at 25° C. to 30° C. under nitrogen. Oxcarbazepine (165 g, 0.654 mol) was added to the reaction mixture. The reaction mixture was heated to reflux (38° C. to 39° C.) and [(S,S)-TsDpen-Ru(p-cymene)Cl] (0.583 g, 0.0009167 mol) dissolved in N,N-dimethylformamide (109 mL) was added to the reaction mixture at 38° C. to 39° C. The reaction mixture was further heated to reflux (40° C. to 45° C.) for about 38 hours and the pH was adjusted to 6 to 7.5 every 3 hours to 6 hours using formic acid (33 mL). After completion of the reaction, dichloromethane (830 mL) and N,N-dimethylformamide (230 mL) were charged at 40° C. to 45° C. After stirring for 15 minutes, 20% aqueous sodium chloride solution (830 mL) was added and stirred for 15 minutes at 40° C. to 45° C. The reaction mixture was cooled to 30° C. to 40° C. and the organic layer was separated. The aqueous layer was again extracted with dichloromethane (330 mL) and the combined organic layer was evaporated under reduced pressure at 40° C. to 50° C. The reaction mixture was cooled (25° C. to 30° C.) and methanol (16.5 mL) was charged followed by the slow addition of toluene (2500 mL). The reaction mixture was stirred for 2 hours at 25° C. to 30° C., filtered, and washed with toluene (330 mL). The solid obtained was suck dried and further dried at 55° C. to 60° C. for 12 hours to obtain the title compound.

Yield: 148.1 g (89.1%)

Chromatographic Purity: 99.24%

Chiral purity: 100%

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A process for the preparation of oxcarbazepine of Formula 1 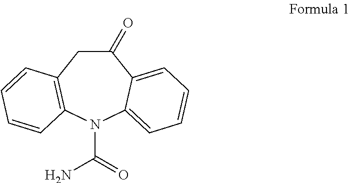 [SMILES: NC(=O)N1c2ccccc2CC(=O)c2ccccc21]
which comprises hydrolysis of 10-methoxy-5H-dibenzo[b,f]azepine-5-carboxamide of Formula 3 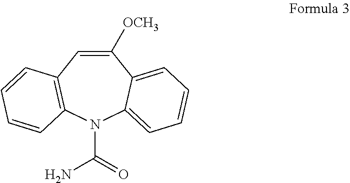 [SMILES: COC1=Cc2ccccc2N(C(N)=O)c2ccccc21]
with an organic acid selected from the group consisting of citric acid, tartaric acid, or mixture thereof.

25. The process according to claim 24, wherein the hydrolysis of 10-methoxy-5H-dibenzo[b,f]azepine-5-carboxamide of Formula 3 is performed in one or more solvents.

26. The process according to claim 25, wherein the solvent is selected from the group consisting of water, esters, aromatic hydrocarbons, halogenated hydrocarbons, ketones, ethers, polar aprotic solvents, and mixtures thereof.

27. (canceled)

28. (canceled)

29. (canceled)

30. (canceled)

31. (canceled)

32. (canceled)

33. The process according to claim 24, wherein the hydrolysis of 10-methoxy-5H-dibenzo[b,f]azepine-5-carboxamide of Formula 3 is performed at a temperature of 30° C. to reflux.

34. The process according to claim 1, further comprising converting the oxcarbazepine of Formula I to eslicarbazepine acetate of Formula A.

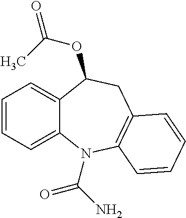 [SMILES: CC(=O)O[C@H]1Cc2ccccc2N(C(N)=O)c2ccccc21]

35. The process according to claim 34, which comprises asymmetric transfer hydrogenation of the oxcarbazepine of Formula 1 in the presence of a catalyst and a hydride source in a mixture of dichloromethane/N,N-dimethylformamide, dichloromethane/water, or dichloromethane/water/methanol to obtain eslicarbazepine of Formula 2; and 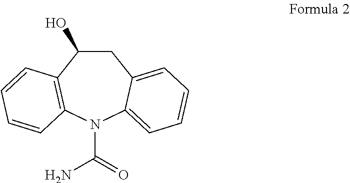 [SMILES: NC(=O)N1c2ccccc2C[C@H](O)c2ccccc21]
acylation of the eslicarbazepine of Formula 2 to obtain eslicarbazepine acetate of Formula A.

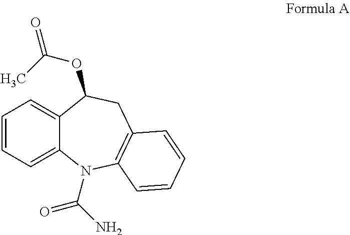 [SMILES: CC(=O)O[C@H]1Cc2ccccc2N(C(N)=O)c2ccccc21]

36. The process according to claim 35, wherein the catalyst is selected from the group consisting of [(S,S)-TsDpen-Ru(p-cymene)Cl], [(S,S)-teth-TsDpen-RuCl], RuCl[(S,S)-FsDPEN](p-cymene), and RuCl[(S,S)-TsDPEN](mesitylene).

37. The process according to claim 35, wherein the molar ratio of the catalyst to oxcarbazepine is from about 0.0005 to about 0.1.

38. The process according to claim 35, wherein the hydride source is selected from the group consisting of sodium acetate/water, formic acid/triethyl amine, potassium-t-butoxide/isopropanol, potassium hydroxide/isopropanol, ammonium formate, and ammonium acetate.

39. The process according to claim 35, wherein the asymmetric transfer hydrogenation is performed in the presence of a phase transfer catalyst.

40. The process according to claim 35, wherein the acylation involves treating the eslicarbazepine with an acylating agent in the presence of a catalyst in one or more solvents at a temperature of 25° C. to reflux.

41. The process according to claim 40, wherein the solvent is selected from the group consisting of water, esters, aromatic hydrocarbons, halogenated hydrocarbons, ketones, ethers, polar aprotic solvents, and mixtures thereof.

42. The process according to claim 40, wherein the acylating agent is selected from the group consisting of acetic anhydride and acetyl chloride.

43. The process according to claim 40, wherein the catalyst is 4-dimethylaminopyridine.
